(19) United States
(12) Patent Application Publication
(10) Pub. No.: US 2023/0059549 A1
(43) Pub. Date: Feb. 23, 2023

(54) CEMENT RETAINING IMPLANT STEM FOR PERMANENT FIXATION

(71) Applicant: WALDEMAR LINK GMBH & CO. KG, Hamburg (DE)

(72) Inventors: Helmut D. LINK, Hamburg (DE); Rodolfo CAPANNA, Firenze (IT)

(73) Assignee: WALDEMAR LINK GMBH & CO. KG, Hamburg (DE)

(21) Appl. No.: 17/757,147

(22) PCT Filed: Dec. 15, 2020
(86) PCT No.: PCT/EP2020/086186

§ 371 Date: Jun. 10, 2022

(30) Foreign Application Priority Data
Dec. 16, 2019 (EP) 19216465.5

(51) Int. Cl. A61B 17/72; A61B 17/88

(52) U.S. Cl. CPC A61B 17/72; A61B 17/88

(57) ABSTRACT

The present invention relates to a stem for or of an implant having a longitudinal axis and at least one recess adapted for cemented implantation into the medullary cavity and/or a cavity created by surgery of a long bone, characterized in that the recess is interrupted by a barrier. Further the present invention relates to a method for preparing a stem of an implant according to any one of the preceding claims for implantation, comprising the step of placing cement into the recess (12) on both sides of the barrier.

### TECHNICAL FIELD

The present disclosure relates to an implant stem comprising a recess interrupted by a barrier and a method for preparing such an implant stem for implantation.

### BACKGROUND OF THE INVENTION

One important objective in orthopedic surgery is a reliable anchorage of an implant within a bone. For implantation into long bones, implant stems are widely used as the primary design element for bone anchorage in a variety of implants. Concerning these implant stems, it is a common technique to anchor implant stems within the medullary cavity of a long bone using cement. An alternative technique is to use an uncemented implant stem that is first anchored during surgery by a press fit and is configured so that bone tissue can grow into the surface structure of the stem resulting in secondary fixation of the stem by bone ingrowth. However, the present invention relates to stems for cemented implantation.

In particular high-risk patients, for example patients suffering from diabetes, overweight and/or cancer, often require additional and instant fixation. Also, the phenomenon of aseptic loosening in these patients is more commonly observed. In such cases, even the technique of cemented implant stems frequently turns out not to be as reliable and as stable as desired.

### SUMMARY OF THE INVENTION

Thus, the objective of the invention is to provide a stem for an implant that offers permanent fixation with respect to fast, reliable and long-lasting stability and that prevents aseptic loosening.

The present disclosure relates to a stem for an implant (and to an implant comprising such a stem), which is preferably adapted for cemented implantation, at least partially, into (parts of) the medullary cavity and/or a cavity created by surgery of a long bone, preferably a human humerus. The stem may also be adapted for bone-ingrowth.

The stem may have a longitudinal axis and comprises at least one recess. Preferably, the recess is formed as a groove and even more preferably as a longitudinal groove, i. e. a groove that extends in the direction of or along the longitudinal axis of the stem. Nonetheless, the recesses may also be formed as holes or have any other shape that allows for incorporating cement.

Preferably, the stem comprises more than one recess, in particular two, three, four, five, or six recesses. The multiple recesses are circumferentially spaced or distributed about the longitudinal axis of the stem. Preferably, they are arranged parallel to each other.

According to the present disclosure, the recess, preferably each recess, is interrupted by at least one barrier. The barrier is oriented transverse to the longitudinal axis of the stem and protrudes from the bottom of the recess radially outwards. As a result, the barrier subdivides the recess into subrecesses along the stem. The barrier is preferably solidly integrated so as to be a part of the stem's shape and material, i. e. the barrier or protrusion forms a part of the stem.

This allows advantageously that cement on both sides of the barrier or protrusion supports fixation in both longitudinal directions of the stem and in the long bone and in particular in the medullary canal. More specifically, the barrier provides the means for establishing a form fit between the barrier and the bone cement in the recess on both sides of the barrier as well as between the bone tissue surrounding the implantation site, in particular cancellous bone tissue, and the implant stem by means of the bone cement. If having an elongated shape, the recess provides said form-fit and enhanced anchorage within the bone and at the same time facilitates removal during revision surgery.

For example, a stem as configured according to one of the embodiments of this disclosure can advantageously be used for anchoring a diaphyseal segment prosthesis, for example after a resection in cancer treatment. In case of a diaphyseal segment prosthesis, permanent fixation is advantageous since a need for replacement of the prosthesis is unlikely compared to other prostheses such as artificial joints. The latter particularly suffer from friction and wear and for this reason may require replacement during the lifetime of the patient.

Preferably, the recess has a minimum width of 1 mm, 2 mm, 3 mm, 4 mm, or 5 mm and a maximum width of 6 mm, 7 mm, 8 mm, or 10 mm. Further, the recess preferably has a depth of up to 0.5 mm, 1 mm, 2 mm, 3 mm, or 4 mm. If the recess has an elongated shape, it preferably has a length of at least 2 cm. Further, the recess preferably does not open up at the proximal or distal end of the stem.

These preferred properties of the recess enhance above-noted form-fit resulting from implantation. Generally, the bigger the recess the better the form fit. However, the biggest gains in anchoring strength are in the lower ranges of above-noted values.

In particular for being adapted for cemented implantation into the medullary cavity of a long bone, the outer contour of the cross section of the stem at the barrier is preferably continuous. In particular, at least one segment of the outer contour of the cross section of the stem at the barrier is round and in particular circular in order to allow smooth insertion into a cavity prepared in a long bone.

For the same reason, at least one segment of the outer surface of the barrier is preferably cylindrical (and, in particular, has a round or circular cross-section) and/or forms a continuous part of an outer surface of the stem outside the recess, which preferably is cylindrical (and, in particular, has a round or circular cross-section).

Further, it is particularly preferred that the whole outer surface of the stem except for the at least one recess is at least along the at least one recess cylindrical (and, in particular, has a circular cross-section). Alternatively, at least a part of the outer surface of the stem and/or the outer surface of the barrier may be tapered with an aperture in a range of up to 10°, 8°, or 6°.

This results in forming a continuous outer surface, in particular with a fully round or circular cylindrical outer surface of the barrier. Such a configuration allows one the one hand a smooth insertion of the stem during the surgical procedure and on the other hand facilitates the preparation of the cavity in the long bone for said insertion.

The stem having a cylindrical outer surface with a circular cross-section and with the at least one recess formed therein further allows for a revision of the stem while preserving bone tissue despite the strong fixation of the stem. In particular, the interface between the circular cylindrical outer surface of the stem and the bone tissue may be released using a core drill. The core drill may be guided by the outer contour while removing material surrounding the stem.

Further, a stem with a cylindrical outer surface increases the strength of the interface between the stem and the bone tissue since the outer cross-section of the stem except for the recesses and preferably the tip of the stem is constant. More specifically, a movement of the stem along the longitudinal axis of the stem is less likely to occur than, for example, of a tapered stem. A movement of a tapered stem out of a bone cavity is easier due to the increase in cross-section of the cavity to its opening, even if the tapered stem has a recess for anchoring the stem within the cavity.

The stem may further be an element of a system of different elements, which are adapted to be mounted to each other in a variety of combinations to respectively form different implants. The stem may also be part of an implant and as such be integrally formed with the shape and material of the implant. For example, the stem may provide anchorage for an artificial joint, wherein the stem is configured to be implanted into a long bone. In particular, the stem may be configured for implantation into the humerus and may form a part of an artificial shoulder joint. In another example, two of the stems may provide anchorage for a diaphyseal segment prosthesis. Here, the diaphyseal segment prosthesis replaces a resected segment of a long bone, wherein the stems are adapted to be implanted into the resected ends of the remaining long bone. The skilled person will appreciate that this may, for example, apply to a humerus or a femur of a long bone.

According to another aspect of the disclosure, a method for preparing a stem of an implant as described above comprises the step of filling the at least one recess on both sides of the barrier with bone cement and placing bone cement on the outer surface of the stem (4) adjacent to the recess (12) before implantation. As a result, the stem (4) is covered with bone cement before being inserted into a cavity of a long-bone. Using cement in this way for fixing the implant within a long bone of a patient ensures a fast and reliable fixation.

### SHORT DESCRIPTION OF THE FIGURES

The following figures illustrate preferred embodiments of the present invention. These embodiments are not to be construed as limiting but merely to enhance the understanding of the invention together with the following description. In these figures, same reference signs refer to features throughout the drawings that have the same or an equivalent function and/or structure. In summary, the figures illustrate the following:

FIG. 1 shows a side view of an implant comprising two stems according to the disclosure;

FIG. 2 shows a perspective view of the implant of FIG. 1 implanted into a human humerus;

FIG. 3 shows an exploded perspective view of the implant of FIG. 1;

FIG. 4 shows a perspective view of an alternative implant assembled using one of the stems of the implant of FIG. 1; and

FIG. 5 shows a perspective view of another alternative implant assembled using one of the stems of the implant of FIG. 1.

### DETAILED DESCRIPTION OF PREFERRED EMBODIMENTS

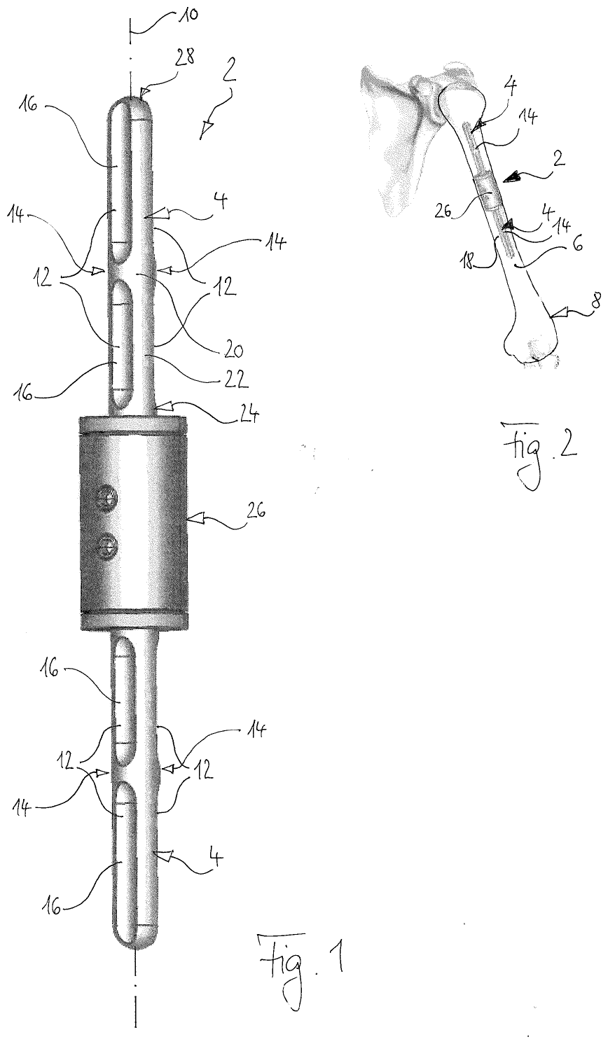

FIGS. 1 to 3 show an implant 2 (namely a diaphyseal segment prosthesis 2) comprising two stems 4 being adapted for cemented implantation into the medullary cavity 6 (after preparing the cavity 6 by surgery) of a long bone and in particular a human humerus 8. The implant 2 has a longitudinal axis 10.

Each of the stems 4 in this exemplary embodiment comprises two recesses 12 in a circumferential direction of the stems 4. Nonetheless any number of recesses 12 in the circumferential direction of above may be formed. The same applies to above-noted dimensions and shap for the recesses 12.

In this exemplary embodiment, the recesses are formed as grooves, i. e. elongated recesses. As illustrated, they may be located parallel to each other, be on opposite sides of the stem and/or extend in the direction of or along the longitudinal axis 10.

Each recess 12 is interrupted by a barrier 14 in the longitudinal direction of the stem 4, which is preferably solidly integrated into the shape and material of the respective stem 4 and in particular about half way along the length of the respective stem. It is also possible to provide more than one barrier along a recess 12, such as two, three, four, or five barriers. Nonetheless, only one or two barriers is preferred.

The at least one barrier allows advantageously that cement 16 on both sides of the barrier 14 supports fixation in both longitudinal directions (against pulling and pushing forces in the humerus 8) by establishing a form fit. This also applies to bone tissue growing into the recesses 12 in case of cementless fixation using a stem 4 that has an outer surface adapted for bone ingrowth.

In case of fixation by bone cement, the form fit is established between the barrier 14 and the cement 16 in the recesses 12 on both sides of the barrier 14 as well as between bone tissue surrounding the implantation site of the stems 4, preferably cancellous bone tissue 18, and the respective stem. In case of cancellous bone tissue, the form fit may be enhanced by causing bone cement to enter into the cancellous bone's surface 18 resulting in a better interlock between the stem and the bone tissue. In case of fixation by bone ingrowth, a form fit is achieved directly between the bone tissue and the stem 4.

In particular for anchoring of a diaphyseal segment prosthesis 2 (e. g. after a resection in cancer treatment), permanent fixation, in particular using bone cement, is advantageous since a replacement of the prosthesis is unlikely compared to artificial joints. In comparison to a diaphyseal segment prosthesis, artificial joints are exposed to friction and wear during everyday use and may thus need replacement during the lifetime of the patient.

If being adapted for cemented implantation into the medullary cavity of a long bone, the outer contour of the cross section of the stem 4 at the barrier 14 is preferably continuous. It preferably has a smooth and even more preferably a polished surface. Further, the outer contour of the stem in cross-section along and except for the recesses 12 and barrier 14 is in particular round or circular (preferably forming a full circle). Likewise, the outer surface 20 of the barrier 14 is in particular round or circular (preferably forming a full circle). As described above, the outer contour of the stem 4 and/or barrier 14 may be cylindrical or tapered.

The outer surface 20 of the barriers 14 preferably forms a continuous part of the outer surface 22 of the stem 4 outside its recesses 12. The outer surface 22 may be, as shown in the exemplary embodiment of the figures, circular cylindrical. In other words, the entire outer surface 22 of each stem 4 outside the recesses 12 and along the recesses is preferably circular cylindrical forming one continuous outer surface with an integrally formed and circular cylindrical outer surface 20 of the barriers 14. Compared to a tapered shape, the cylindrical shape has no influence on the depth of the recesses 12.

As discussed above, a circular cylindrical surface of the stem 14 is particularly preferred since it provides on the one hand a strong anchoring within the bone tissue (by cemented implantation or bone ingrowth) and on the other hand allows for a revision of the stem while preserving bone tissue, in particular using a core drill. Such a core drill may be guided by the circumferential surface of the stem 14 while removing material (bone tissue and/or cement) surrounding and being in contact with the stem. As a result, the material anchoring the stem 14 within the bone cavity is interrupted and the stem 14 may easily be pulled out of the cavity. As described above, these advantages are provided by the shape of the stem 14 being generally cylindrical instead of being tapered.

Consequently, the basic shape of the stem 14 is preferably cylindrical and even more preferably circular cylindrical. The basic shape is defined by the shape of the outer surface of the stem except for the at least one recess 12, the distal end 28, and the connective end 24.

Each stem 4 comprises one connective end 24 adapted to be mounted to a diaphyseal segment body 26 and one continuously integrated preferably rounded and in particular semi-spherical distal end 28. The latter prevents damage to bone tissue during insertion.

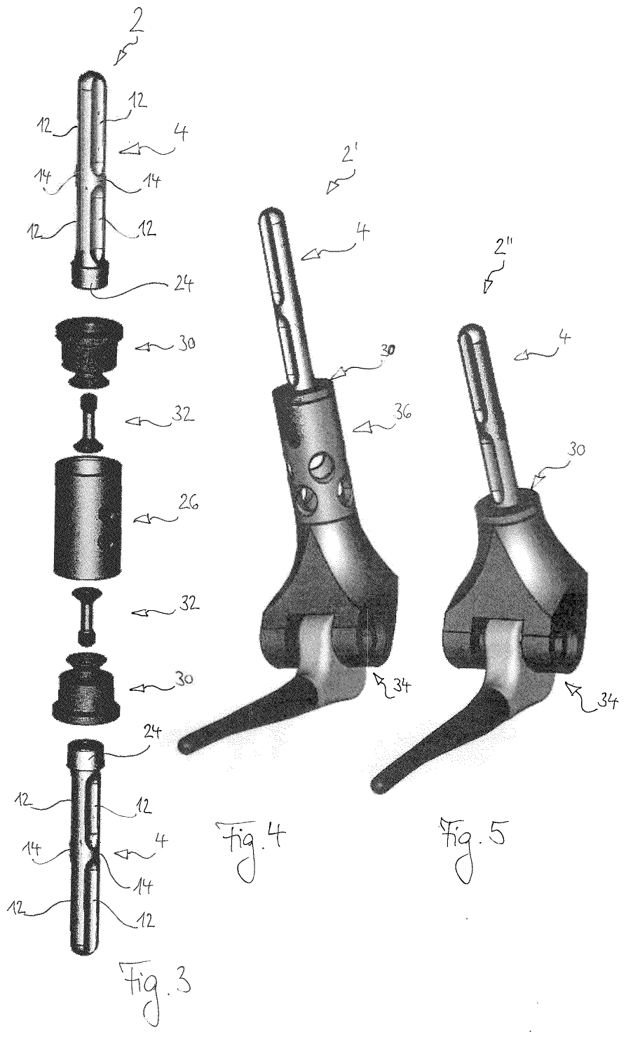

As shown in the exemplary illustrative embodiments of the figures, the stems 4 are members of a system of different elements like the diaphyseal segment body 26, the adapter 30, or the connective screw 32 (cf. FIG. 3). These elements are adapted for being mounted to each other in a variety of combinations to respectively form different implants 2, 2′, 2″.

For example, FIGS. 4 and 5 show implants 2′ and 2″, respectively, wherein the stem 4 is mounted to provide anchorage for an artificial elbow joint. The implants 2′ and 2″ are mounted as a combination of a variety of elements like the stem 4, adapter 30, and connective screw 32 also shown in FIG. 3, and additionally an elbow joint replacement 34 and a segment 36 for replacing a section of a long bone.

### REFERENCE SIGNS

2, 2′, 2″ implant
4 stem
6 medullary cavity
8 human humerus
10 longitudinal axis
12 recess
14 barrier
16 cement
18 cancellous bone
20 outer surface of barrier 14
22 outer surface of stem 4 outside the recess 12
24 connective end
26 diaphyseal segment body
28 distal end
30 adapter
32 connective screw
34 shoulder joint group
36 joint adjacent segment

1. A stem for or of an implant, the stem being adapted for implantation into a cavity of a long bone, wherein the stem has a longitudinal axis and at least one recess, wherein said at least one recess is interrupted by at least one barrier.

2. The system or implant of claim 1, wherein the recess is formed as a groove, preferably a longitudinal groove.

3. The system or implant of claim 1, wherein the barrier is solidly integrated into the shape and material of the stem.

4. The system or implant of claim 1, wherein the recess has a minimum width of 1 to 5 mm and a maximum width of 6 to 10 mm.

5. The system or implant of claim 1, wherein the recess has a depth of 0.5 mm to 4 mm.

6. The system or implant of claim 1, wherein the outer contour of the stem at the barrier is continuous.

7. The system or implant of claim 1, wherein the outer contour of the stem at the barrier is circular.

8. The system or implant of claim 1, wherein the outer surface of the barrier is cylindrical.

9. The system or implant of claim 1, wherein at least a segment of the outer surface of the barrier forms a continuous part of an outer surface of the stem outside the recess.

10. The system or implant of claim 7, wherein at least one segment of the outer surface of the barrier and the outer surface of the stem outside the recess are cylindrical.

11. The system or implant of claim 1, wherein the geometry of the stem is adapted to be at least partly implanted into the medullary cavity of a humerus.

12. The system according to claim 1, wherein the system has different elements adapted to be mounted together in a variety of combinations to form respective different implants.

13. An implant comprising a stem according to claim 1.

14. A method for preparing a stem of an implant of claim 1 for implantation, comprising the step of filling the at least one recess on both sides of the barrier with bone cement and placing bone cement on the outer surface of the stem adjacent to the recess.
